(19) United States
(12) Patent Application Publication
(10) Pub. No.: US 2019/0232562 A1
(43) Pub. Date: Aug. 1, 2019

(54) PROCESS OF MANUFACTURE OF CUSTOMIZED SPLIT INSOLE FOR DIABETIC PATIENTS

(71) Applicant: Rangasamy Naidu Educational Trust, Peelamedu (IN)

(72) Inventors: Rajesh Ranganathan, Coimbatore (IN); Sivakumar Ganesan, Coimbatore (IN)

(21) Appl. No.: 15/881,885

(22) Filed: Jan. 29, 2018

(51) Int. Cl. B29C 64/393; A43B 17/00; A43B 7/22; A43B 17/14; A43D 39/00; B33Y 50/02; B33Y 70/00; A61F 5/14; G16H 10/20

(52) U.S. Cl. CPC B29C 64/393; A43B 17/003; A43B 7/223; A43B 7/226; A43B 17/14; B33Y 10/00; B33Y 50/02; B33Y 70/00; A61F 5/14; G16H 10/20; A43D 2200/60; A43D 39/00

(57) ABSTRACT

Under the inventive process, the customised split insole is made by processing manually collected medical data and arch data along with plantar pressure distribution data and foot type data both of which are collected using a computer enabled Plantar Pressure Measuring Device in a first computer and processing these data in a second computer to generate a Projection Data which is expressed in Shore Hardness Value. Then, ranking of the projection data into a zone wise Ranking Index, determining the elasticity of the material to be used for the said Shore Hardness Value, converting the said shore hardness value into Surface Tessellation Language and transmitting the same to a third computer built into a 3D printing machine which selects a suitable material and its elasticity zone wise as per the Shore Hardness Value and 3D prints the same.

### FIELD OF INVENTION

The present invention pertains to the process of manufacturing customized split insole for use by diabetic patients. The customized split insoles so made would eliminate or reduce the rate of diabetic foot ulcer formation, gangrene and the requirement for amputation in diabetic patients. It also increases the comfort of the foot during static and dynamic conditions.

### BACKGROUND OF THE INVENTION

According to International Diabetes Federation (IDF, 2015), 415 million people have diabetes in 2015. This is expected to rise to 642 million by 2040. A study on the diabetic foot: global view conducted by (Boulton 2005) suggested that globally diabetic foot ulceration is a major healthcare challenge, the factors do not vary country wise. In India inappropriate footwear or barefoot gait were the common cause for neuropathic foot problems (Boulton 2005).

Foot insole are a type of foot orthoses. They improve the function of the human foot, which provide support and distribution of individual's body weight. For the reduction of plantar pressure diabetic footwear and orthotic insoles are used, Investigations on the selection of insole material properties, indicated that there is no optimum material for the development of insole but, indicated that parameters like body mass and physical activity of the individual affect soft tissues in the plantar surface. According to (Ledoux et al. 2013), location of ulceration caused by elevated plantar pressure is unclear, so they conducted the study on the effect of location-specific plantar pressures. During the study, it was found that overall mean peak plantar pressure was higher for subjects with diabetic foot ulcer, and varied location wise and from person to person.

In order to understand high foot pressure and ulceration, Boulton et al., (1983) employed optical pedobarograph. They conducted a study using three groups, one without diabetes, the other with diabetes but without neuropathy and the third with diabetes and with neuropathy. Their study evaluated the relationships among foot pressures, neuropathy and foot ulceration. Boulton et al., (1983) study revealed that, abnormal high foot pressures existed for diabetic neuropathy patients compared to the diabetic control patients. Furthermore, high pressures are found at ulcerative sites for patients who had previous history of foot ulceration. With ulceration occurring at sites of high plantar foot pressures, it was found that, reduction of foot pressure lead to reduction of foot ulceration in diabetic neuropathic patients.

Duckworth et al. (1985) working on plantar pressure measurement identified that foot ulcer occur at high pressure regions, and inorder to detect them they suggested static and dynamic pressure measurement devices to measure plantar surface pressure points. In order to measure plantar pressure distribution, Wolfe et al., (1991) suggested, ‘Plantar Pressure Measuring Device’ which is clinically used to scrutinize the asymmetry of plantar pressure distribution in young adults with ankle fractures. They also suggested the use of the same device to measure plantar pressure in diabetic patients with Charcot neuroarthropathy. They used these measures to correct the gait pattern in accident victims, to analyse orthotic problems only and not for any other purpose.

Dombroski et al. (2014) in their work used scanning tools to obtain the geometry of the human foot and used additive manufacturing technique to develop the custom foot orthosis.

These customised foot orthosis were used for manufacture of artificial limbs and prosthetic products and not for any other problems they were not used for making insoles. Study conducted on gait pattern revealed that, each individual has his/her own style of walking which is an outcome of the gait pattern developed based on various factors such as height, weight mannerism, psychological behaviour, way of live, body mechanism, injury, accidents or other factors. Similarly, the gait pattern of a person will undergo changes from child hood, adolescence, middle age and old age depending on various factors including the above said factors.

The gait pattern of a person has a lot to do with the stress and pressure exerted on the foot. Therefore, the gait pattern directly affects the plantar surface of the foot. In normal person, from childhood to middle age the gait pattern does not have any significant harmful effect on the plantar surface of the foot. However, ageing may cause certain orthotic problems which may directly or indirectly affect the plantar surface of the foot. The build-up of pressure on the plantar foot surface has a direct bearing a plantar foot ulcers, especially in diabetic patients. Foot Ulcers occurs at sites of the high pressure on the plantar surface of the foot.

The human foot consists of three parts namely the hind foot, mid foot and the fore foot. The hind foot consists of two bones one on top of the other. The mid foot consists of five bones packed close together, while the fore foot consists of five metatarsals each with phalanges (toes).

The superior surface of the hind foot forms the ankle joint which articulates with the tibia and fibula in the medial and lateral position respectively.

During walking, the entire body weight is mostly borne by one leg at a time. The fibula bone transmits weight to the talus and to the rest of the foot. During walking, when the body first touches the ground, the calcaneus (Head bone) takes the entire weight. But, however, still some body weight is shared by the other foot as well. Once the heal is firmly on the ground, the other foot leads the ground. The fore foot touches the ground, but usually, the lateral border of the foot takes on the weight first transmitting it through the cuboid bone and the base of the fifth metatarsal. Immediately, thereafter, the whole foot is on the ground. Thus, the body weight is transmitted from the calcaneus or head bone to the cuboid bone to the base of the fifth metatarsal and then the heads of all the five metatarsals. Then, when the other foot swings forward, the heal begins to leave the ground and the whole weight is shifted to the forefoot. Thereafter by a strong contraction of the toes, the body is pushed forward to transfer its weight on the other foot which is now in a stable position to receive the transmitted weight. This is one walking cycle. Therefore it can be said that in one walking cycle, when the foot comes down, it rests back of the heal, then on the lateral side of the metatarsal and finally on the heads of the metatarsal to push off for the next step.

Similarly, when a person is standing, both foot are on the ground where the foot is like an arch sparing the mid foot from weight bearing.

It is therefore fascinating to analyse how instinct the mechanism of walking or standing or running is. The foot therefore displays several motions like acceleration, deceleration, sudden stop, jumping, twisting, turning, kicking, squatting and so on. During all these motions, the pressure exerted on the plantar foot varies.

In diabetic patients, there are combination of various adverse factors which affect the strength and health of the plantar foot. The end result of these various risk factors are neuropathy and tissue damage.

So unlike normal patients, the foot of a diabetic patient is not prepared to handle the pressure build-up on various areas of the plantar foot during various motions in view of its un-healthy state caused due to the said risk factors. Therefore, build-up of plantar pressure at particular areas of the plantar foot will aggravate the neuropathy or tissue damage thus leading to ulcer formation. One way to avoid or reduce the damages of such ulcer formation is by distributing the pressure build-up on the plantar foot to other non-pressure build-up areas by suitably off-loading the pressure

That apart, During leg movement, there is a interlink articulation of one or more of these bones which exerts pressure at various levels on the plantar surface of the foot. Therefore, the gait pattern of a person directly affects the pressure exerted on various areas of the plantar foot surface through the foot bone leading from the leg bone. It varies from person to person depending on his gait pattern and is not uniform for all. The pressure exerted on the various areas of the plantar surface of foot is called plantar pressure. This can be measured by a sensory device and processed by linking it to a computer.

Diabetic patients have a lot of foot related problems. The simplest formation which can lead to greatest complication and loss of limb is the formation of foot ulcers. It is known that have a reduced healing rate when diabetic patients compared to normal healthy persons. So the formation of a foot ulcer tends to progresses ultimately leading to gangrene and amputation of the phalanges or any other part of the foot or the leg itself in extreme cases.

Irrespective of the cause of formation of foot ulcer in diabetic patients, the exertion of pressure on the various areas of the plantar surface of the foot directly affects ulcer formation. This pressure exertion on the various areas of the plantar surface of foot is based on the gait pattern of the person.

When measuring the plantar pressure of a person, it can be seen that there are many pressure build-up areas on his foot. In some of these areas, the pressure build-up is very high. These areas are more prone to foot ulcer formation. By off-loading these pressure in the pressure build-up areas in particular in the peak pressure build-up areas, ulcer formation can be eliminated if not reduced. Off-loading of the pressure build-up is done by dissipating the build-up pressure to the surrounding areas of the plantar surface so that one particular point is not exposed to the pressure build-up which is the cause of ulcer formation.

Further, in diabetic patients, in view of neuropathy or internal tissue damage due to lack of blood supply or deficit blood supply, capillaries get clogged, leading to pressure build-up which aggregates the chances of tissue injury and ulcer formation. So all the more it is necessary to off-load the pressure build-up on the plantar foot surface of diabetic persons to avoid foot ulcers.

More than 15% of the ulcer results in amputation of foot or limb. The treatment of plantar ulcers is also very complex. So the prevention by off-loading of the plantar pressure surface by using customised insoles is a very effective remedy. The inventive customised split insole addresses this issue in a technically better and high cost effective manner as described herein.

Therefore peak pressure in plantar foot surface of diabetic persons is a common route cause for diabetic foot ulcer, leading to gangrene and amputation. To overcome this problem, many insoles for diabetic patients have been designed the world over. However, these insoles are subject to the following limitations;
a. Are not made to individual requirement.
b. The entire customised insole is of single piece.
c. The elasticity and Shore Hardness Value is uniform throughout the entire insole and not split zone wise.
d. Though there is cushioning effect there is no offloading of pressure build up.
e. The problem faced by diabetic patients and Doctors is that the present insoles are not very effective in distributing.
   Off-loading the plantar pressure build-up because their elasticity, thickness and density is uniform throughout the insole. Whereas, under the inventive split insole, the elasticity, density and thickness of the insole material is varied zone wise so as to very effectively off-load the pressure build-up, than in conventional insoles.
f. Some of the customised insoles are ‘jelly type’ to provide cushioning effect. But, the softness of the jelly is uniform throughout. This has two disadvantages namely that it reduces the grip during dynamic activity and secondly, it does not effectively offload the pressure build-up in the pressure points.
g. In the present invention, the material hardness varies between the pressure build-up points and the other areas so as to ensure effective off-loading, of the build-in pressure which is also achieved by effectively increasing the plantar contact area of the foot with the floor.
h. The customised single insoles do not consider the arch support in the foot. Because of which, the pressure distribution to the mid foot is not addressed. The present invention addresses this aspect by specifically considering the arch data and the type of foot in calculating the thickness and Shore Hardness Value of the material to be used for the arch.
i. The loss of material is negligible, therefore reducing the material and processing costs. Therefore, the present invention addresses these limitations and short comings and ensures effective off-loading of pressure built-up in the pressure areas by varying thickness and Shore Hardness Value of the material zone wise for peak pressure areas in a zone wise.
j. Replacement cost is high as the entire insole has to be replaced, whereas in the product under the inventive process only the worn-out part needs to be replaced.

Further, none of the existing insoles were split insoles focused towards effectively offloading the peak pressure zone.

### SUMMARY OF THE INVENTION

The inventive process pertains to the manufacture of customized split insole for persons with diabetic problems. Firstly, requisite data is collected by manual or technical means. This is then processed in a separate computer to produce an output which is a set of instruction in ‘Surface Tessellation Language’. This instruction is fed to another computer built into a 3D printing machine which prints the customized split insole as per requirement.

The manual data consists of medical data, in particular the foot size and the foot arch data. The technical data consists of plantar pressure distribution data and foot type data which are collected using a sensor pad of a device and then stored in a connected computer (First computer).

From the pressure distribution data the best bare foot ‘Trial Walk’ is selected manually. This manual selection along with the foot type data is then transmitted to the second computer. Similarly, the manually collected medical data and arch data are also directly fed to the second computer. The foot template (foot size) is also manually selected in the second computer from a preprogrammed inbuilt data base. The second computer then processes all the above said data and the selected foot template to generate the projection data which is expressed as Shore Hardness Value of the material to be used. The projection data is then converted into ‘Surface Tessellation Language’ which is a set of machine language instruction.

The said ‘Surface Tessellation Language’ instruction is then transmitted to the third computer which is inbuilt into the 3D printing machine which ultimately prints the customized split insole as per requirement.

### BRIEF DESCRIPTION OF THE DRAWINGS

FIG. 1 is a process sequence.

FIGS. 2A to 2C are a person walking on the pressure sensor pad.

FIG. 3 is plantar pressure distribution and foot type.

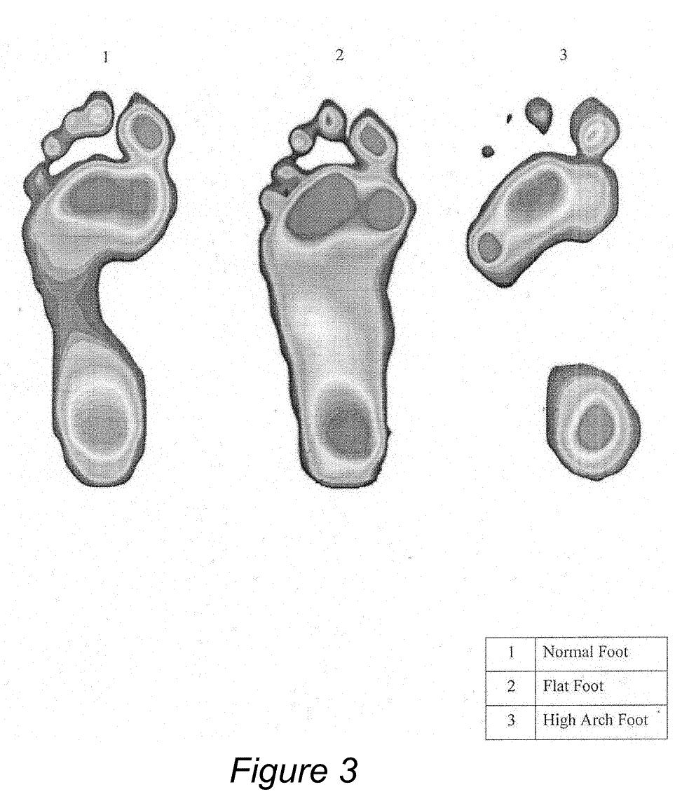

FIG. 4 is a plant pressure distribution, best trial walk.

FIG. 5 is an arch measuring foot board.

### DESCRIPTION OF THE INVENTIVE PROCESS

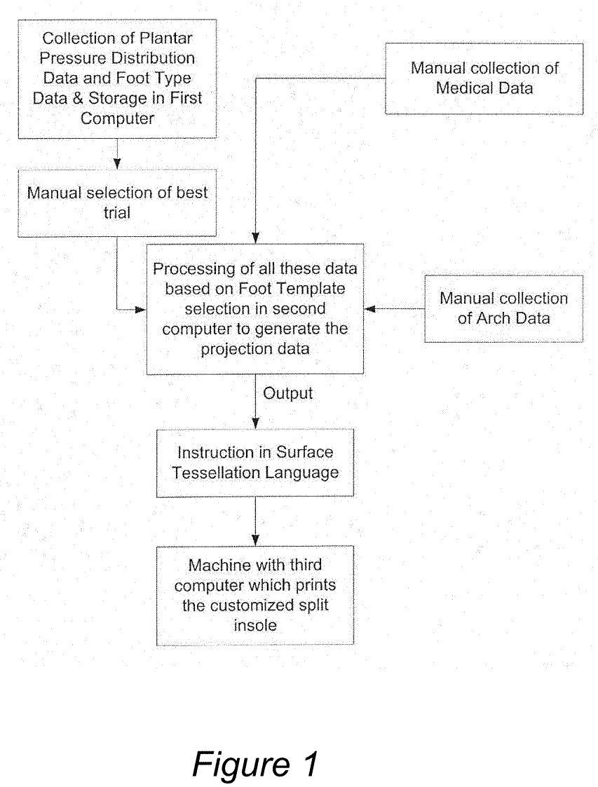

The process of making a customized split insole under the present inventive process is as described in FIG. 1. Under this inventive process, various data pertaining to the foot of the diabetic patient is collected manually and technically, and processed to get the projective data based on which the hardness of the material and type of material to be used is determined, and then the customized split insole printed by the machine designed for such purpose. The following types of data are collected and processed;
i. Medical data—which is manually collected by the technician by a one to one interaction with the patient. This data serves to identify the individual for whom the insole is made and to bring on record the patients name, age, height, weight, foot size and general or specific animus as applicable to the patient.
ii. Plantar pressure distribution data—this data pertains to the pressure exerted by the patients' foot on the floor. This data is collected by making the patient walk bare foot for four or five times on the sensor pad of a special equipment and stored in the first computer, and then selecting the best ‘Trial Walk’ data manually.
iii. Foot Type Data—is the data pertaining to the type of foot of the patient i.e. whether normal, flat or high arch foot. This data is also collected when the patient walks four or five times on the sensor pad of the special equipment. This data so collected is also stored in the first computer.
iv. Arch data—This data pertains to the type of foot i.e. normal, flat or high arch foot and is obtained by physical measurement of the foot using an ‘Arch Measuring Foot Board’.

The selected plantar pressure distribution data and the foot type data which are collected by the sensor pad and stored in the first computer are transmitted to the second computer.

The arch data collected manually is also directly fed into the second computer. The foot template is manually selected in the second computer.

The second computer processes the selected plantar pressure distribution data and, the foot type data received from first computer, the arch data, selected foot template and generates the projection data which is then converted into ‘Surface Tessellation Language’ in the second computer itself. This forms the instruction basis on which the insole is to be made. This data determines the type and hardness of the material to be used in each zone of the split insole, and the nature of projections.

The ‘Surface Tessellation Language’ instruction from the second computer is transmitted to the third computer which is built into a 3D printing machine which prints the customized split insole as per the instruction received from the second computer, by choosing/selecting the appropriate material of requisite Shore Hardness Value.

### Working of the Inventive Process

The foot profile of each individual is different, in terms of size, shape, plantar pressure distribution and gait pattern. Therefore a uniform material composition for foot insole is not suitable. This is because each individual's peak pressure point and gait pattern will vary. The design of the split insole is made according to an individual's foot type and plantar pressure distribution, so as to dissipate the pressure build up on the peak pressure points.

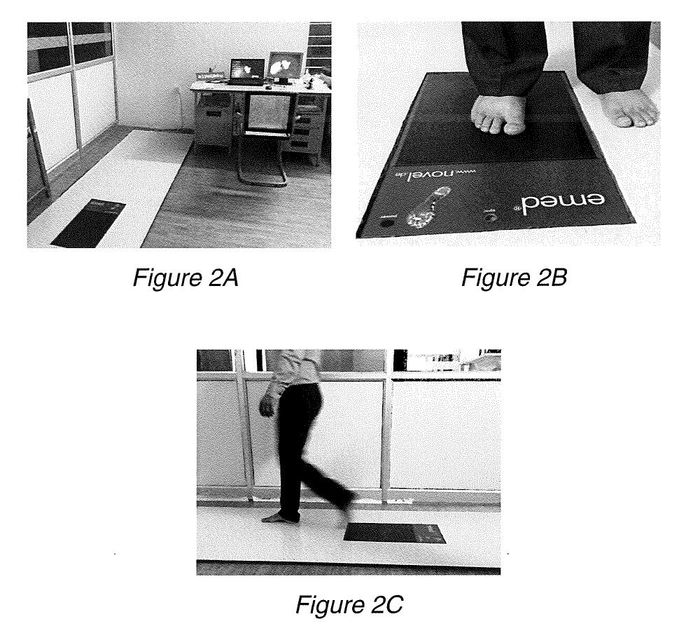

In this inventive process, the medical data of the person such as, his name, age, height, weight and foot size are collected manually. Then the person for whom the customized foot insole is to be made is made to walk on a ‘Plantar Pressure Measuring Device’ as in FIG. 2. The measurement of plantar pressure using the ‘Plantar Pressure Measuring Device’ provides the distribution of force over the plantar surface of the foot and thus provides detailed information specific to each region of the foot that can be separately addressed. The person is made to undergo trial walk on bare foot for four to five times or more, on this device. During which time the data on plantar pressure distribution is captured. The foot type viz. normal, flat or high arch foot is also captured by this device. All the captured data are stored in a computer (first computer) as in FIG. 1.

The best ‘Trial Walk’ is manually ascertained from the four or five trials or more and stored in the first computer. The ‘best Trial Walk’ is ascertained by considering the gait pattern (in particular gait pattern at heal strike and toe off) and consistency of plantar pressure distribution, the total contact area comprising of the peak pressure, distribution in all the trials, individual zone peak pressure and plantar pressure distribution pattern on the plantar surface. By considering these factors, the best trial walk is manually selected and sent to the second computer along with the data on the foot type.

However, any abnormality in the gait pattern, ambiguity at heal strike and toe-off, any consistency in peak pressure and contact area, non-similar pressure patterns, abnormal contact area and abnormal plantar pressure are not considered in selecting the best ‘Trial Walk’, because that is not his normal walking style/gait pattern.

### Example 1

A total of four trial walks were performed on each foot and the same were compared to select the best trial walk for the design purpose. By comparing the four trials abnormal gait pattern is found in trials 1, 2 and 4 in the metatarsal region. Ambiguity in the gait pattern is found in heel strike as in trial 4. Variation in the contact area and peak pressure consistency are found in trials 2 and 4. Abnormal peak pressures 375 and 710 kPa is found in the trial 2 and 4 respectively, so the trial 1, 2 and 4 were excluded and trial 3 is selected for designing the customized split insole.

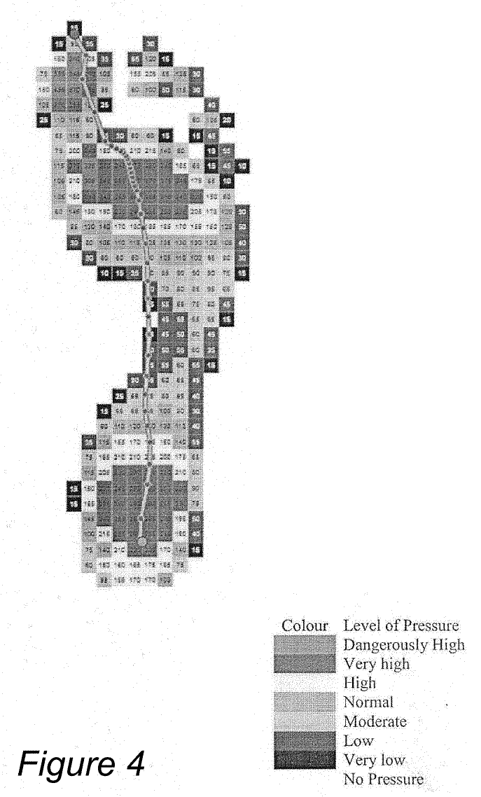

The trial walks which are stored in the first computer are stored as graphical representations. Thus, the ‘Plantar Pressure Measuring Device’ collects the plantar pressure data stored in the first computer which analyses the same based on the plantar pressure exerted on the contact areas of the foot and the ground and represents it geometrically and graphically in pre-dimensional manner as in FIG. 4. The best Trail Walk and the foot type are then manually selected and transferred to the second computer. The arch data taken separately is also fed manually into the second computer. The Foot Template is selected from the set of pre-stored sizes in the second computer. The second computer then processes all the data and gives the projection data.

The best Trial Walk data which is manually selected from the trial walks as in FIG. 4 is called ‘Plantar Pressure Data’. The plantar pressure distribution on the manually selected best trial walk is as shown in FIG. 4. The report of pressure build-up is indexed in various colours with the pressure units indicating the pressure build-up and the pressure reading. The same is as analysed in Table 1 below:—

TABLE 1
Analysis of FIG. 4

| S. No | Colour | Pressure Build up | Pressure Rating |
|---|---|---|---|
| 1 | Pink | 300 kPa and above | Dangerously high |
| 2 | Red | 220 kPa to 299 kPa | Very high |
| 3 | Yellow | 150 kPa to 219 kPa | High |
| 4 | Green | 100 kPa to 149 kPa | Normal |
| 5 | Light blue | 60 kPa to 99 kPa | Moderate |
| 6 | Dark blue | 30 kPa to 59 kPa | Low |
| 7 | Black | 10 kPa to 29 kPa | Very low |
| 8 | White | | No |

From the above Table-1 which is an analysis of FIG. 4 it can be seen that the areas highlighted in pink, red and yellow are peak pressure build up areas and are more vulnerable to nerve and tissue damage and the chances of ulcer formation is very high. More is the area of pink colour, the chances of ulcer formation is highest and once formed ulcer would be very severe and chances of healing are very low, which directly increase the chance of gangrene formation and amputation.

Therefore, pink and red colour is directly proportional to high risk ulcer formation, and poor healing and thereby leading to high possibility of gangrene formation.

The projections offload the pressure build up in the pink, red and yellow areas to the other normal, moderate or low pressure areas so as to offload the pressure build up in the said areas. From FIG. 4 and Table-1 it is seen that the pressure build up is more in the metatarsal region (53 cm2), heel region (40.5 cm2). It is moderate or above moderate in the phalanges except the big toe and normal in the lateral foot and negligible in the midfoot. As regards the big toe (1st phalange) the pressure build up varies depending on the gait pattern.

Using the medical data collected from the patient, the appropriate pre-programmed foot template is manually selected in the second computer, to suit the opted foot size. The template is the size of the foot which is preset for various foot sizes, ranging from Size 6 to 11, which are the mostly used templates for this purpose.

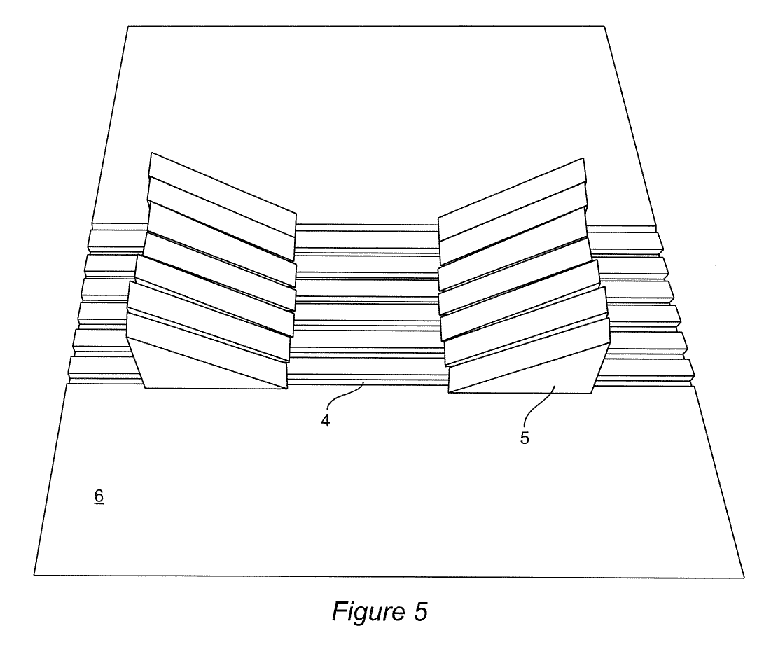

The Arch Data is collected separately and physically using a device called ‘Arch Measuring Foot Board’, which consists of a measuring board, 7 grooves, and 7 triangular wooden pegs, on each side of the grooves, as in FIG. 5. The arch data is collected depending on the foot type i.e. normal, flat and high arch foot. The significance of providing arch support is that it will help in offloading the peak pressure across different anatomical zones and increases the surface area of contact of the foot with the ground.

The person places his foot on the foot board in between the two set of triangular wooden pegs. Then for normal and flat foot persons, the pegs on the side of the foot arch are adjusted by bringing them close to the foot and the arch reading taken. For high arch foot persons, the pegs on both sides of the foot are adjusted by bringing them close to their respective side of the foot and the readings taken. The data so collected is manually fed directly to the second computer.

Then the second computer processes the plantar pressure distribution data and foot type data as received from first computer along with the arch data directly fed into the second computer and based on the foot template manually selected generates the projection data. The projections data ultimately enables even distribution of pressure on the plantar surface of the foot, by suitable projections, as required to offload pressure buildup in the various areas of the plantar surface and to increase the plantar contact area with the insole.

For generation of the projection data, the plantar surface of the foot is ranked into six zones for normal and flat foot persons. For persons with high arch foot, it is split into five zones. The pressure points in the plantar surface are ranked zone wise based on a ‘Ranking Index’. For each Ranking Index, Shore Hardness Value for the corresponding elasticity is as furnished in Table 2 below:

TABLE 2
Material and shore hardness value

| Ranking Index | Shore Hardness Values |
|---|---|
| 1 | — |
| 2, 3, and 4 | Shore Hardness Value A40 |
| | and A50 |
| 5 | Shore Hardness Value is A60 |

From the table, it is seen that the zone wise pressure point ranking is given with reference to the Ranking Index and the elasticity is expressed in terms of Shore Hardness Value.

The Ranking Index of table 2 shows that where the index is ‘1’ the pressure build-up is very high implying that these are areas of peak pressure build-up. As the Ranking Index value decreases, the pressure build-up decreases. For the maximum ranking index value, the elasticity of the insole material to be used at that particular zone is to be maximum, implying that the Shore Hardness Value by which it is expressed is to be minimum.

$$\begin{matrix}\begin{matrix}Ranking Index Value α Elasticity\end{matrix} \\ ( or ) \\ \begin{matrix}Ranking index value α \frac{1}{Shore Hardness Value}\end{matrix}\end{matrix}$$

It can therefore be seen that if the ranking index is more, then the pressure acting on that point is more, in which case, the elasticity is also more and the Shore Hardness Value is less and vice-versa.

Now, the shore hardness value for each zone is arrived at. Therefore, the net result of the process of all the said data by the second computer is the projection data expressed in Shore Hardness Value for the various zones of the customised split insole. These Shore Hardness Value is now converted by the second computer into the machine readable instruction in ‘Surface Tessellation Language’ and transmitted to the third computer which forms part of the machine which 3D prints the customised split insoles.

The machine used for 3D printing is the Polyjet Technology Objet Connex Machine which functions using addictive manufacturing technique. This machine is operated by a built in third computer. The third computer based on the input instruction received from the second computer in ‘Surface Tessellation Language’ selects the appropriate material to be used for 3D printing based on the shore hardness value received.

The material used is VeroClear RGD810, TangoPlus FLX930 of Shore Hardness Value A40, A50 and A60 for normal foot persons and flat foot persons respectively and is VeroClear RGD810, TangoPlus FLX930 of Shore Hardness Value A40 and A60 for High arch persons. The selection of material for the appropriate shore hardness value and appropriate foot type for each zone is as furnished in Table 3. The third computer chooses the quantity and type of material and its thickness to suit the Shore Hardness Value for each zone and 3D prints the customized split insole.

TABLE 3

| Shore Hardness | | |
|---|---|---|
| Value | Material | Type of Foot |
| | TangoPlus FLX930 | Normal, Flat and High Arch foot |
| A40 | VeroClearRGD810, | Normal, Flat and High Arch foot |
| | TangoPlus FLX930 | |
| A50 | VeroClearRGD810, | Normal, and Flat Arch foot |
| | TangoPlus FLX930 | |
| A60 | VeroClearRGD810, | Normal, Flat and High Arch foot |
| | TangoPlus FLX930 | |

### Advantages of the Present Inventive Process

1. No separate dies, templates or fixtures are required as in conventional models,
2. The time taken to make the product is less than that of any other insole manufacturing process,
3. Inventory holding cost of raw material is drastically reduced,
4. Wastage of raw material is negligible in the present invention whereas it is quite high in conventional manufacturing processes.
5. Replacement cost is very low
6. Life of insole is high as only worn out parts need by replaced and not the entire insole.
7. Effective distribution of plantar pressure is achieved.

We claim:

1) The process of manufacturing a customised split insole by processing;
a. manually collected Medical Data,
b. Plantar Pressure Distribution Data, and Foot Type Data collected using a computer enabled Plantar Pressure Measuring Device in a first computer, and
c. manually collected Arch Data,
in second computer where the Medical Data and Arch Data are directly manually fed into the second computer and converting the output projection data into a zone wise “Ranking Index”, and determining the elasticity of the material based on the ‘Ranking Index” and in turn allotting the Shore Hardness Value of the material zone wise, and then converting the said Shore Hardness Value into Surface Tessellation Language and transmitting it to a machine built third computer, which selects the suitable material and, the elasticity of the material, zone wise, as per the Shore Hardness Value and 3D prints the customised split insole.

2) An inventive process as in claim 1, where Medical Data consists of the name, age, height, weight, foot size, general or specific animus applicable to person.

3) An inventive process as in claim 1, where the Plantar Pressure Data is captured by 4 or 5 bare foot Trail Walks made by the person on the Plantar Pressure Measuring Device and stored in the First Computer whereupon the best Trail Walk Data is manually selected and sent along with the foot type data also captured by the Plantar Pressure Measuring Device and stored in the First Computer, to the Second Computer.

4) An inventive process as in claim 1, wherein the arch data as recorded using Foot Arch Measuring Board is manually fed into the second computer and the Foot Template is manually selected in the second computer from pre-stored Foot Template Data.

5) An inventive process as in claim 1, wherein the second computer processes the Pressure Distribution Data, Foot Type Data, Arch Data and the selected Foot Template, to generate the projection data plotted as a ‘Ranking Index’.

6) An inventive process as in claim 1, where the elasticity of the material to be used is determined based on the ‘Ranking Index’ and the corresponding Shore Hardness Value is assigned to it.

7) An inventive process as in claim 1 where the Shore Hardness Value is uniform throughout a zone, depending on the peak pressure points on the plantar surface of the foot and the corresponding elasticity of the material for that particular plantar surface of the zone, but varies zone-wise.

8) An inventive process as in claim 1, where the Shore Hardness Value is converted into Surface Tessellation Language and transmitted to the Third Computer which in turn is built in to Polyjet Technology, Objet Connex Machine.

9) An inventive process as in claim 1, where the Third Computer selects the material which is VeroClear RGD810, TangoPlus FLX930, and the quantity of material to be used zone wise corresponding to the Shore Hardness Value of each zone which is A40, A50 and A60 respectively for normal and flat foot persons and 3D prints the customised split insole accordingly.

10) An inventive process as in claim 1, where the Third Computer selects the material which is VeroClear RGD810, TangoPlus FLX930, and the quantity of material to be used zone wise corresponding to the Shore Hardness Value of each zone which is A40 and A60 for high arch persons and 3D prints the customised split insole accordingly.
